(19) United States
(12) Patent Application Publication
(10) Pub. No.: US 2023/0142125 A1
(43) Pub. Date: May 11, 2023

(54) NOVEL MICROSTRUCTURES OF MYCELIUM AND MYCELIUM-BASED MATERIALS

(71) Applicant: MycoWorks, Inc., Emeryville, CA (US)

(72) Inventors: Philip Ross, San Francisco, CA (US); Rachel Linzer, Albany, CA (US); Matthew Scullin, San Francisco, CA (US); Ritu Bansal-Mutalik, Albany, CA (US); Ivana Brzonova, San Francisco, CA (US); Jordan Chase, El Cerrito, CA (US); Csaba Kotsmar, Oakland, CA (US)

(21) Appl. No.: 17/796,374

(22) PCT Filed: Feb. 3, 2021
(86) PCT No.: PCT/US21/16437

§ 371 Date: Jul. 29, 2022

Related U.S. Application Data
(60) Provisional application No. 62/969,636, filed on Feb. 3, 2020.

(51) Int. Cl. C12N 1/14; C08L 5/08; C12N 1/38

(52) U.S. Cl. CPC C12N 1/14; C08L 5/08; C12N 1/38

(57) ABSTRACT

A novel microstructure of a mycelium material exhibiting a high mass density whereby the mycelium material exhibits a much greater spatial density of hyphal branches and hyphal connections than is otherwise found in nature. The material further provides improved macroscopic properties typified by high tensile strength, high flexural toughness and increased tear strength.

### RELATED APPLICATIONS

This application is a 35 U.S.C. 371 National Stage Entry of International Application No. PCT/US2021/016437 filed Feb. 3, 2021 and claims the benefit of US provisional patent application 62/969,636, filed Feb. 3, 2020, the disclosure of which is incorporated herein in its entirety.

### BACKGROUND OF THE DISCLOSURE

### Technical Field of the Disclosure

The present invention relates in general to mycelium, and more particularly to mycelium and other mycomaterials exhibiting improved microstructure and macrostructure characteristics.

### Description of the Related Art

The fungal life cycle has been increasingly understood and manipulated by modern industry and science. It has a long history of being useful for food and medicine, and it is known that given the appropriate conditions, fungal tissue can quickly be amplified to commercially useful volumes. Many of these techniques now present in the art relate to the alteration of growth of the vegetative mycelium's expanding hyphae—the constituent, threadlike cells that make up mycelium—through stimuli of a gravitropic, thermotropic, thigmotropic, phototropic, chemotrophic, and/or hydrotropic nature. For instance, through the altering of subtle factors that affect mycelial growth, it is possible to alter and direct fungal hyphae, mycelium and tissue to express a range of variably determined physical characteristics.

More than just promoting random high volume growth, fungal tissue growth may be directed and controlled such that the resultant mycelial tissue presents additional utility for its use in various industries. For instance, the resultant product, upon harvest, may be cured and finished to take on qualities that are similar in texture, look and performance to plastics, foams, or animal skins. A common use for these materials includes industries in which leather would conventionally be used.

In its natural state and natural mode of growth, mycelia encompass and/or infiltrate organic matter that acts as a food source. For instance, in FIG. 1, a Ganoderma, a saprotrophic fungus that feeds on lignocellulose from dead and downed wood, is fruiting from a tree stump as is known in nature. The visible fruiting bodies of the fungus are growing out of the mycelium network that is intercalated with the wood. Other fungi are primarily subterranean in nature, and may integrate with a medium of soil, mulch, or a mass of wood chips. However, these types of mycelium are closely related to those used in industry as a source of raw material, wherein they are grown (fermented) within a food media, and the food media is integrated into the very microstructure of the mycelium material that is harvested, processed, and industrialized. In addition to the fruiting bodies, there is a vegetative mycelium component, which is the subject of this application.

Similar prior art materials and methods comprise vegetative mycelium, or of mycelium and a second material. Said materials are typically composed of a microstructure of mycelium that is intermingled and combined with its solid food media. Prior art FIG. 2 depicts one such example, where the mycelial network is grown around a secondary material that may be its food source. By creating a woven mycelial mesh of mycomaterial and other structures, material properties of the resultant mass can be improved beyond that found in mycomaterial alone. Along these lines, it is known that several methods have been developed for producing mycelium bio-composite materials. However, such mycelium materials typically have low spatial density and fail to provide adequately improved mechanical properties such as high tensile strength, high flexural toughness and increased tear strength required for certain applications.

Although the current state of the art does provide a raw material for use in many industries, and while through the use of a woven or mesh-like hybrid of mycomaterial and other materials can create improved structures, there remains a need to further improve the raw mycomaterial alone. Further, there is a need for the material to not only match but to in some cases exceed the physical properties of conventional industrial materials (leather, plastics etc.) that the improved mycelial material aims to replace.

There is thus a need for a vegetative mycelium material having novel microstructure and macrostructure characteristics. The present application discloses pure mycelial networks grown following specific protocols to change and/or manipulate the microstructure and thus enhance mechanical properties, and in some cases these mycelial networks are also combined with secondary materials. Such a novel mycelium material exhibits a much greater spatial density of hyphae, hyphal branches and/or hyphal connections, and thereby improved macroscopic properties typified by high tensile strength, high flexural toughness and increased tear strength. Such a myco-material preferably when used as a composite exhibits a high tensile strength and a reduction in average pore size and in some embodiments reduced water absorption capability as compared to natural materials and state of the art mycelial composites.

Such a needed material would in some cases be pure mycelium without any solid media particulate, and in others the material may be a composite, such as a mycelial network incorporating a textile. As will be apparent from a reading of the present specification, the material described herein overcomes the shortcomings in the industry that matches and, in some cases, exceeds conventional industrial materials.

### Summary of the Disclosure

To minimize the limitations found in the prior art, and to minimize other limitations that will be apparent upon the reading of the specification, the present application provides a novel vegetative mycelial material exhibiting properties not found in conventional industrial materials. The novel material comprises a composition exhibiting an increased mass density of the mycelium material such that it exhibits a much greater spatial density of hyphal branches and hyphal connections than conventional or natural mycomaterials. Improved macroscopic properties typified by high tensile strength, high flexural toughness and increased tear strength are all exhibited by these materials, and all supported by the increased material density achieved in the present application.

A first objective of the present invention is to provide a vegetative mycelium material having novel microstructure and macrostructure characteristics.

A second objective of the present invention is to provide a mycelium material that exhibits a much greater spatial density of hyphal branches and hyphal connections than are otherwise found in conventional or natural mycomaterials, and thereby improved macroscopic properties typified by high tensile strength, high flexural toughness and increased tear strength.

A third objective of the present invention is to provide a mycelium material with the hyphae density increased with respect to conventional mycomaterials.

A fourth objective of the present invention is to provide a mycelium material having a tensile strength of at least 8 MPa or above.

A fifth objective of the present invention is to provide a pure mycelium material without any added solid media particulate, the pure mycelium exhibiting small average pore size, low water absorption properties, and high bally flex.

A sixth objective of the present invention is to provide a mycelium/textile composite material exhibiting novel physical characteristics and attributes.

These and other advantages and features of the present invention are described with specificity so as to make the present invention understandable to one of ordinary skill in the art.

### BRIEF DESCRIPTION OF THE DRAWINGS

In order to enhance their clarity and improve the understanding of the various elements and embodiment shown herein, the figures have not in all cases been drawn to scale. Furthermore, elements that are known to be common and well understood to those in the industry are not depicted in order to provide a clear view of the various embodiments of the invention, thus the drawings are generalized in form in the interest of clarity and conciseness.

FIG. 1 is a prior art image of a Ganoderma fungal species fruiting from a tree stump in nature;

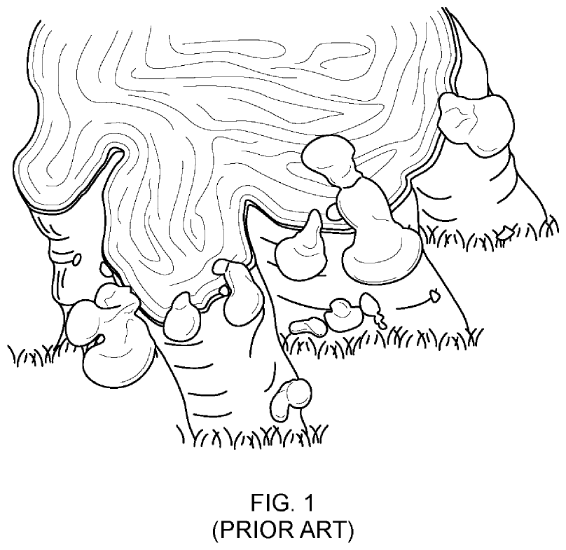

FIG. 2 is a prior art image of a myco-material composite wherein the fungal material has intermixed with material that may be its food source;

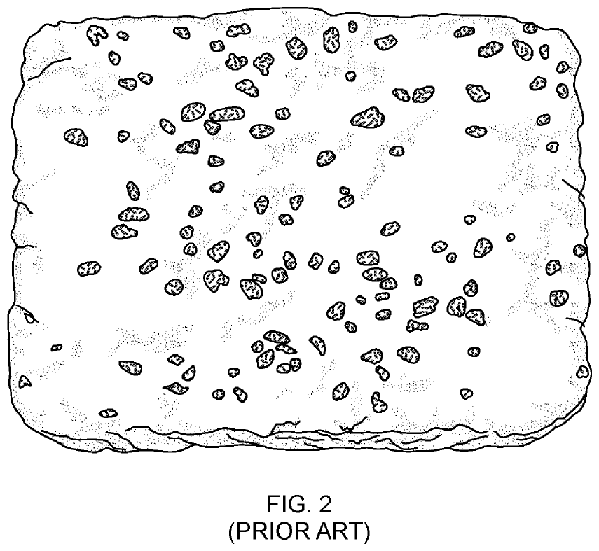

FIG. 3 is a representative prior art image of a cultivated mycelial network.

FIG. 4 is at the same scale as FIG. 3, and shows a prior art image of the state of art of conventional mycomaterial;

FIG. 5 is at the same scale as FIGS. 3 and 4 and depicts a mycelial network grown without the specific process underlying the material claimed in this invention;

FIG. 6 is at the same scale as FIGS. 3-5 and shows dense hyphal packing in the mycelium material in accordance with the one embodiment of the invention;

FIG. 7 is at lower magnification as compared to FIGS. 3-6 and shows an entire cross section of an alternative embodiment of the present invention of pure mycelium wherein the mycelial composition comprises high-density mycelial layers combined with further introduction of through-plane features;

FIG. 8 is a stress (MPa)—strain (% elongation) curve for a sheet of mycelium material in accordance with the present invention;

FIG. 9 depicts prior art data concerning conventional mycomaterials generally; and

FIG. 10 is at the same scale as FIGS. 3-6 and shows a scanning electron micrograph depicting pore sizes.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized, and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

As used herein, the singular forms “a”, “an” and “the” include plural referents unless the context clearly dictates otherwise. “And” as used herein is interchangeably used with “or” unless expressly stated otherwise. As used herein, the term ‘about” means +/−5% of the recited parameter. All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “wherein”, “whereas”, “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

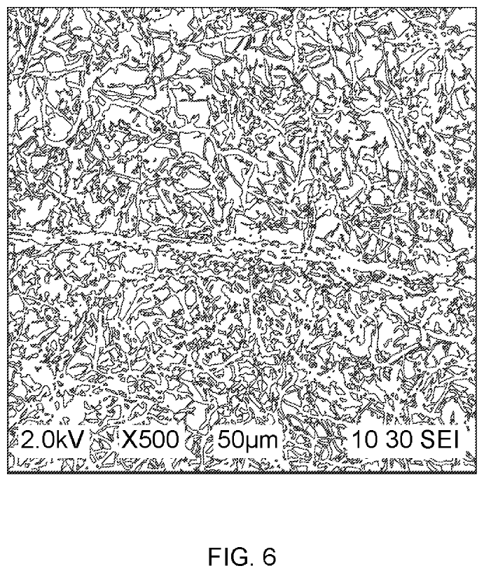

Mycelium is the vegetative component of the fungal material, and comprises along other components chitin and polysaccharides. It is a biological material that may grow into vast, interconnected networks, creating numerous branches per unit volume and potentially spanning vast distances and dimensions. Utilizing such growth characteristics, combined with controlled conditions of growth, the preferred embodiment of the present invention, as illustrated in FIG. 6, describes a new type of mycelial material that exhibits improved spatial density of hyphal branches and hyphal connections, and thereby improved macroscopic properties typified by high tensile strength, high flexural toughness and increased tear strength, which are all supported by the increased material density achieved in the present application.

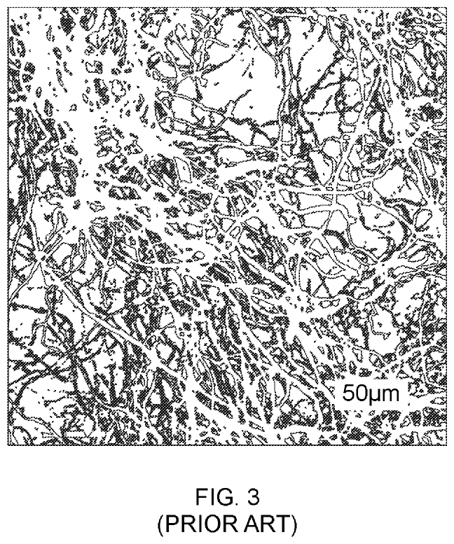

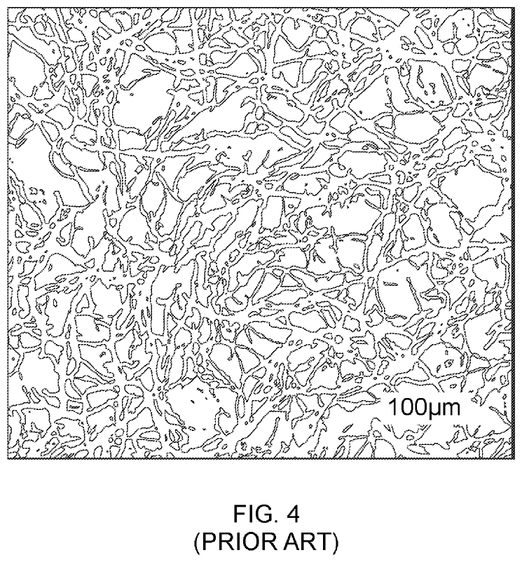

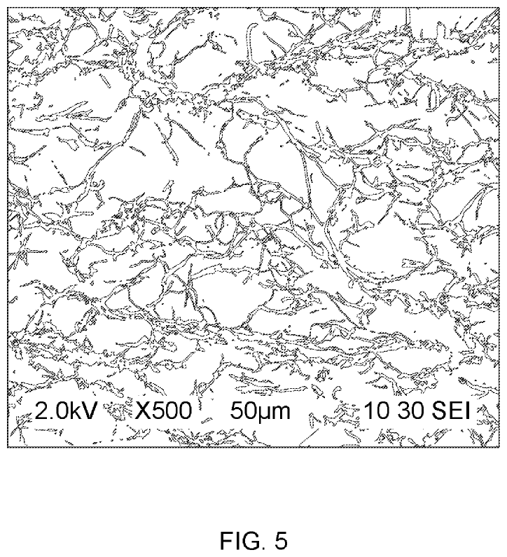

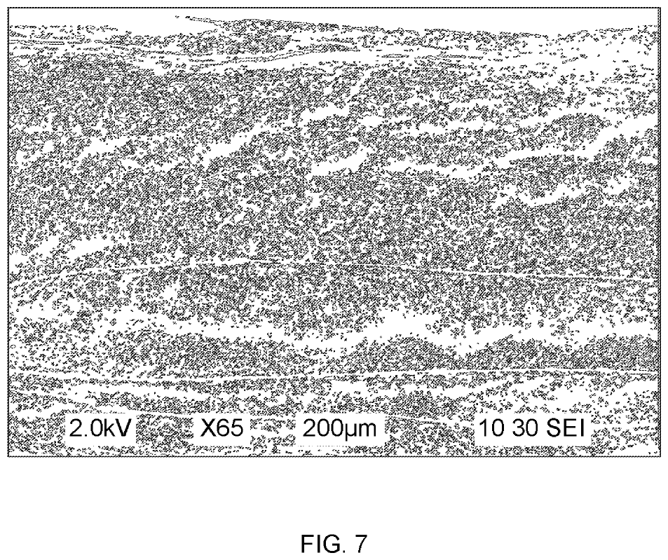

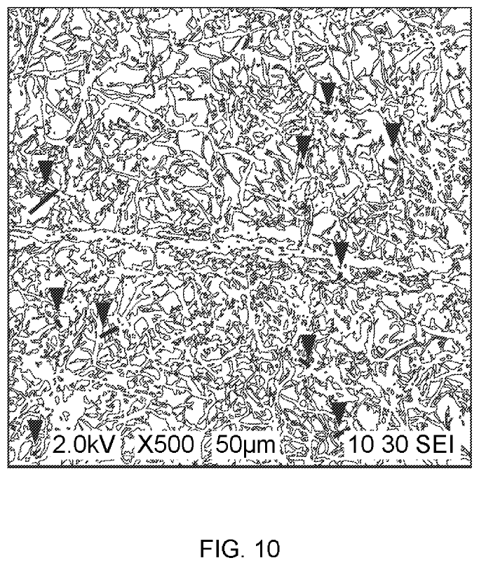

In FIG. 6, the preferred embodiment of the present invention discloses a mycelial material comprising increased hyphal density. It is clearly visible that the density of hyphae present on FIG. 6 is higher compared to FIGS. 3-5 . The scale is preserved among these four figures as well as FIG. 10 to be discussed later. FIG. 4 is a prior art image of the typical density of hyphae found in conventional mycomaterials. The dense hyphal packing in the mycelium material of the preferred embodiment of the present invention is illustrated in FIG. 6 and FIG. 7 and shows dense hyphal packing. Advanced microscopy like SEM, TEM, cryoTEM, Tomography, X ray and the like reveal that the morphology of natural mycelium, which comprises a filamentous network of branching, interconnected tubes that encompass and infiltrate a solid media that is then concurrently digested and/or metabolized.

As shown in FIG. 3, the morphology of plated mycelium (in this case, a wood decay fungus in the family Ganodermataceae) reveals lower, less regularly packed features as compared to the unique mycelium microstructure shown in FIG. 6, and is also directly integrated with its nutritional media (it is not pure mycelium). Furthermore, this mycelium microstructure does not exhibit the increased number of branched connections to nearest-neighbor hyphal branches when compared to the novel mycelium disclosed herein and shown in FIGS. 6 and 7. The scale bar is preserved for direct comparison of FIG. 3 through FIG. 6. In FIG. 6 it is shown the microstructure is inherently higher density than other growth forms, as well as pure in so much as there is only the preferred fungal mycelium in the cross section (no solid media particulate).

The mycelium microstructure of the present invention is shown in FIG. 6. The development of through-plane features such as those shown in FIG. 6 further contribute to the enhanced qualities of the present invention. The improved mycelial materials may be further prepared via the periodic application of external stimuli to the growing mycelium during the fermentation process. Via the application of periodic manipulations during the growth phase, the hyphae may be grown into predetermined directions such that they are arranged orthogonally, as specific angles, into lattices or other two-dimensional and three-dimensional organizations.

FIG. 7 represents an alternative embodiment of the present invention wherein the non-textile mycelial composition comprises unique layerings of the high-density mycelial combined with further introduction of through-plane features.

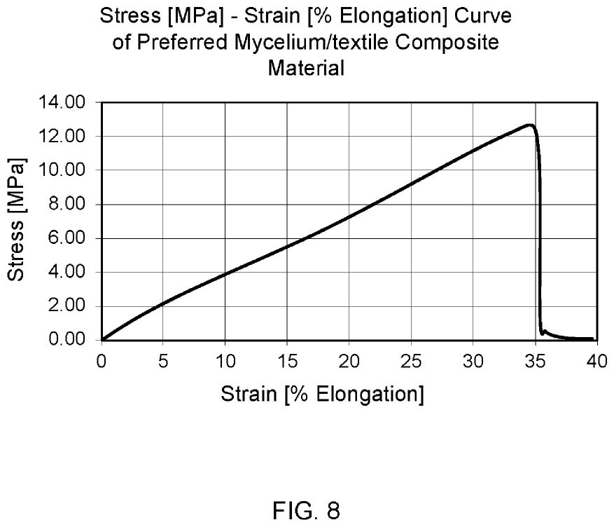

Mechanical analysis and macroscopic sample-testing demonstrates improved strength and durability properties of the mycelium-microstructure, including but not limited to ultimate tensile strength & elongation, tear strength, reduction in water absorption and average pore size (hyphae per unit area). For instance, FIG. 8 depicts a strain curve for a sheet of mycelium wherein the final break of the preferred material occurs at about 12.7 MPa.

While the composite material disclosed herein is a composite mycelial material of mycelium and an additional material or textile, such as cotton, nylon, or felt, pure mycelium in a preferred embodiment composes at least 50% of the net weight of the composite. These non-fungal materials are incorporated by the mycelium to form a bio-composite with the mycelial compounds. In another embodiment, pure mycelium makes up at least 60% of the dry weight of the composite. In other embodiments, pure mycelium makes up at least 75% of the composite and in still further embodiments 90%. In certain embodiments with a larger share of textile, the weight percent of mycelium maybe less than 50%, such as at least 40%, at least 30%, at least 20%, or between any of those values and 50%.

In these embodiments, the elongation prior to breakage may be above 35%. Thus, the present invention can be said to describe a mycomaterial having on a macroscale (see definition below) on the order of at least 2×, at least 4×, at least 5×, at least 10×, or at least 20× that of the state of the art.

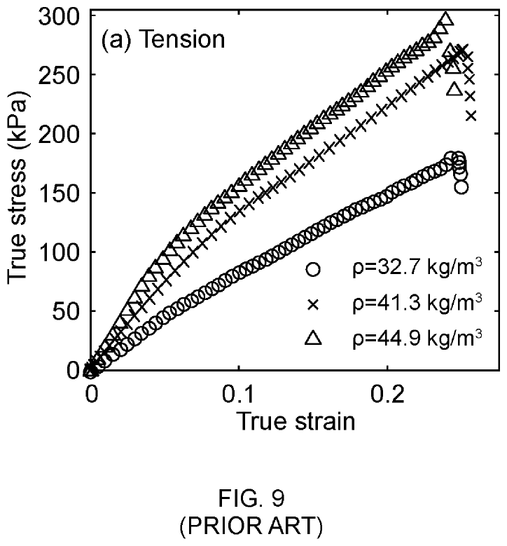

Turning to FIG. 9, the true strain of prior art mycomaterials on a macroscale is shown. For instance, under tension, the elongation may reach somewhere on the order of 25% of original length (true strain 0.25) prior to failure. In a preferred embodiment of the present invention containing pure mycelium, the elongation under strain may reach 38% or even higher in a preferred embodiment. In other embodiments, the amount exceeds 70%, or exceeds 85%, or exceeds 100%. In less preferred embodiments the amount exceeds 50%. Tensile strength at final failure for materials in this particular prior art shown on FIG. 9 is at the maximum of 300 kPa. In an alternative embodiment of the invention, the pure mycelium material due to the higher density of hyphae has a tensile strength value at final failure of 3.1 MPa (3100 kPa) or above.

An additional advantage of the present invention is related to porosity and pore size distribution. FIG. 10 depicts one such method for measuring material average pore size. Here, pore size is captured on electron micrographs and for each micrograph, a subset of pores in the surface plane is selected using randomly generated X,Y coordinates, with the longest dimension of each pore is measured. The average pore size area may be calculated as well. As shown in FIG. 10, longest pore length of ten randomly chosen pores is marked by a white line. Large arrowheads above each line are added for the sake of clarity for this application. Greater sample sizes, such as 100 or 1000 pore size values may be taken in order to obtain more accurate real world averages.

In one embodiment, the vegetative mycelium material has a high spatial density of hyphal branches and hyphal connections and an average pore size of between 3.0-6.0 um. In another, the vegetative mycelium material has an average pore size of between 3.5-5.0 um. It is known that the prior art exhibits an average pore size of between 12 and 15 or higher, with specific measurements in one instance equaling 12.2 and 14.65 um. In one embodiment, the material exhibits an order of magnitude reduction in hyphae pr unit area/pore size distribution as compared to the state of the art.

The decreased average pore size exhibited in the improved mycomaterial leads to additional characteristics such a greatly reduced water absorption capability of the present mycelia as compared to conventionally grown mycomaterials. It is known that conventional mycelia may absorb anywhere from 500% to 2000% of its weight in water. In preferred embodiments herein, the vegetative mycelium disclosed herein exhibits a water absorption capability is at most 150% of its weight in water. In other words, in this embodiment 1 kg of material will absorb at most 1.50 kg water. During the process of absorbing water, the thickness of the material increases, in one case at most 40% and in another case at most 30%. In a third case the thickness increases between 30-40%. In less preferred embodiments the vegetative mycelium material water absorption capability is at most 125% of its weight in water, and in still further embodiments is between 125% and 150% of its weight in water.

With respect to tensile strength, given here in MPa, it is noted that the strength of the vegetative mycelium material may preferably be at least 8 MPa, but in less preferred embodiments may be at least 12 MPa, and in still further less preferred embodiments may be between 8 MPa and 12 MPa.

With respect to tear strength, given in N, it is noted that the strength of the vegetative mycelium material may preferably be at least 8N, but in less preferred embodiments may be at least 60 N, and in still further less preferred embodiments may be between 8 N and 60 N. In still further embodiments the tear strength of the material may be at least 100N, or between 8N and 100N.

With respect to bally flex, the vegetative mycelium material in some embodiments exhibits a bally flex of at least 100,000, while in other embodiments exhibits a bally flex of at least 150,000, while in still further embodiments, it exhibits a bally flex of at least 200,000. In one example, Bally flex is measured using an bally flex tester such as that available by Schap Inc. of Spring Lake, Mich., United States of America. In this embodiment method ASTM D6182 was employed, which bends a strip of material 22.5 degrees, from 90 degrees, to 67.5 degrees and back at 100 cycles per minute at ambient temperatures.

In each of the embodiments described herein as a composite or textiled material, such embodiments are not composed of pure mycelium, however, the weight of the mycelium as a fraction of the composite is known to make up at least 80% of the weight of the composite. In some instances, this is 80% of the net weight of the composite, while in others it is 80% of the dry weight of the composite. In specific cases, such as composite sheets of mycelium and cotton, the wt % of mycelium as a fraction of net weight was 91.17%, or 81.77% as a wt % mycelium of dry weight. In another example of composite lmm sheets of mycelium and felt, the wt % of mycelium as a fraction of net weight was 89.28%, or 79.80% as a wt % mycelium of dry weight.

For the above example and all examples in this case, the quantified measurements (tensile strength, water absorption, etc.) are exemplary of the mycomaterial on a macro scale. That is to say the characteristics involved may not necessarily be unique as compared to one or two strands of cells viewed at a microscopic level. Rather, the quantified attributes described in this document are found on large format mycelia blocks or sheets. For instance, they could be expected on a 12 inch long by 12 inch wide sheet that is between 0.5 mm and 20 mm thick, or a 1 inch by 1 inch sheet that is between 0.5 mm and 20 mm thick, or a piece of mycomaterial having a length, width, height, or any combination of those on the order of hundreds of microns. For instance, a sheet of mycomaterial on the order of hundreds of micrometers thick would exhibit the quantified characteristics described here over vast lengths or widths, thus providing a tremendous advantage as a raw material for further processing by myriad industries.

It is also important to note that the testing herein is for mycelium on a macroscale is in some embodiments without fabric or other composite features. It is known in the art that to improve the material characteristics of mycomaterial for use by industry that the material may be made as a composite, in some cases combined with other non-mycomaterials. In some cases, materials described herein are combined in this way. In some cases, the materials described herein are lubricated with for instance glycerin. In some instances, the mycomaterial is processed similarly to other analogous materials in industry, such as cowhide leather as shown in Table 1, below.

Prior art mycelium materials are of demonstrably lower and/or limited mechanical properties and qualities as compared to those of the present invention. Similarly, state-of-the-art materials do not combine a sufficient degree of strength with a practical level of flexural capability (‘stretchiness’, ‘bendability’, etc.); therefore, the unique microstructure of mycelium in this disclosure represents a new paradigm of mycelium material properties that are enabled by the way it is grown, in order to create an improved microstructure of mycelium.

TABLE 1

| | | | | Cowhide |
|---|---|---|---|---|
| Test | Example A | Example B | Example C | Leather |
| Tensile strength (MPa) | 5.6-7.4 | 8.8-9.3 | 9.2-10.2 | 8.0-25.0 |
| Elongation (%) | 16-36% | 55-80% | 51-52% | 10-80% |
| Tongue tear strength (N) | 6.7 | 52.6 | 9.9 | >20 |
| Stoll abrasion (cycles, 1 lb) | >1,300 | >1,300 | | >1,300 |
| Bally Flex (cycles) | >20,000 | >100,000 | | >10,000 |
| Colorfastness to distilled water | 4.5 | | 4 | 4.5-5 |
| (1-5 rating, 5 is high) | | | | |
| Colorfastness to sea/salt water | 4.5 | | 4 | 4.5-5 |
| (1-5 rating, 5 is high) | | | | |
| Colorfastness to perspiration | 4.5 | | 4.5 | 4.5-5 |
| (1-5 rating, 5 is high) | | | | |
| Colorfastness to water spotting | 5 after | | 5 after | 4.5-5 |
| (1-5 rating, 5 is high) | drying | | drying | |
| Colorfastness to solvent wicking | 5 | | 3.5-5 | 4.5-5 |
| (1-5 rating, 5 is high) | | | | |
| Colorfastness to crocking | 5 dry, | | 4 dry | 4.5-5 |
| (dry and wet) | 4 wet | | | |
| (1-5 rating, 5 is high) | | | | |
| Colorfastness to machine washing | One wash: | | Not | Not |
| (1-5 rating, 5 is high) | slight change | | recommended | recommended |
| Colorfastness to UV exposure | 1.5 | | 4.5 | 5 |
| (1-5 rating, 5 is high) | | | | |

The foregoing description of the preferred embodiment of the present invention has been presented for the purpose of illustration and description. It is not intended to be exhaustive or to limit the invention to the precise form disclosed. Many modifications and variations are possible in light of the above characterization. It is intended that the scope of the present invention to not be limited by this detailed description, but by the claims and the equivalents to the claims appended hereto.

We claim:

1. A vegetative mycelium material at least 10 mm by 10 mm by 0.5 mm in size, the vegetative mycelium material grown as a component of a fungal organism under controlled growth conditions and comprising:
a. a mycelial microstructure further comprising mycelial compounds having a mass density and comprising chitin and polysaccharides;
b. whereby the vegetative mycelium material has a high spatial density of hyphal branches and hyphal connections, and thereby improved macroscopic properties of flexural strength exhibited by a bally flex of at least 100,000;
c. whereby the vegetative mycelium material is pure mycelium with no solid media particulate.

2. The vegetative mycelium material of claim 1 wherein the material exhibits a bally flex of at least 150,000.

3. The vegetative mycelium material of claim 1 wherein the material exhibits a bally flex of at least 200,000.

4. A vegetative mycelium material at least 10 mm by 10 mm by 0.5 mm in size, the vegetative mycelium material grown as a component of a fungal organism under controlled growth conditions and comprising:
a. a mycelial microstructure further comprising mycelial compounds having a mass density and comprising chitin and polysaccharides;
b. whereby the vegetative mycelium material has a high spatial density of hyphal branches and hyphal connections and an average pore size of between 3.0-6.0;

5. The vegetative mycelium material of claim 4 wherein the vegetative mycelium material exhibits an average pore size of between 3.5-5.0.

6. The vegetative mycelium material of claim 4 wherein the material is a vegetative mycelium composite textile formed between the fungal organism and non-fungal materials.

7. The vegetative mycelium material of claim 4 whereby the vegetative mycelium material is pure mycelium with no solid media particulate.

8. The vegetative mycelium material of claim 4 wherein the vegetative mycelium material water absorption capability is at most 150% of its own weight in water.

9. The vegetative mycelium material of claim 9 wherein the vegetative mycelium material water absorption capability is at most 125% of its own weight in water.

10. The vegetative mycelium material of claim 9 wherein the vegetative mycelium material water absorption capability is between 125% and 150% of its own weight in water.

11. A vegetative mycelium composite textile at least 10 mm by 10 mm by 0.5 mm in size, the vegetative mycelium material grown as a component of a fungal organism under controlled growth conditions and comprising:
a. a mycelial microstructure further comprising mycelial compounds having a mass density and comprising chitin and polysaccharides;
b. non-fungal materials forming a composite textile with said mycelial compounds;
c. whereby the vegetative mycelium composite textile has a high spatial density of hyphal branches and hyphal connections, and thereby improved macroscopic properties of high water retention, tensile strength, flexural strength, and tear strength;
d. whereby mycelial material makes up at least 50% of the weight of the composite; and
e. wherein the vegetative mycelium composite textile exhibits a tensile strength of at least 8 MPa.

12. The vegetative mycelium composite textile of claim 11, wherein the mycelial material makes up at least 50% of the net weight of the composite.

13. The vegetative mycelium composite textile of claim 11, wherein the mycelial material makes up at least 50% of the dry weight of the composite.

14. The vegetative mycelium composite textile of claim 11, whereby the composite exhibits a tensile strength of at least 12 MPa.

15. The vegetative mycelium composite textile of claim 11, whereby the composite exhibits a tensile strength of between 8 and 12 MPa.

16. A vegetative mycelium composite textile at least 10 mm by 10 mm by 0.5 mm in size, the vegetative mycelium material grown as a component of a fungal organism under controlled growth conditions and comprising:
a. a mycelial microstructure further comprising mycelial compounds having a mass density and comprising chitin and polysaccharides;
b. non-fungal materials forming a composite textile with said mycelial compounds;
c. whereby the vegetative mycelium composite textile has a high spatial density of hyphal branches and hyphal connections, and thereby improved macroscopic properties of high water retention, tensile strength, flexural strength, and tear strength;
d. whereby mycelial material makes up at least 50% of the weight of the composite; and
e. wherein the vegetative mycelium composite textile exhibits a tongue tear strength of at least 8N.

17. The vegetative mycelium composite textile of claim 16, wherein the mycelial material makes up at least 50% of the net weight of the composite.

18. The vegetative mycelium composite textile of claim 16, wherein the mycelial material makes up at least 75% of the net weight of the composite.

19. The vegetative mycelium composite textile of claim 16, wherein the mycelial material makes up at least 50% of the dry weight of the composite.

20. The vegetative mycelium composite textile of claim 16, wherein the mycelial material makes up at least 75% of the dry weight of the composite.

21. The vegetative mycelium composite textile of claim 16, whereby the composite exhibits a tongue tear strength of at least 50 N.

22. The vegetative mycelium composite textile of claim 16, whereby the composite exhibits a tongue tear strength of between 8 and 50 N.
